(19) United States
(12) Patent Application Publication
(10) Pub. No.: US 2017/0184695 A1
(43) Pub. Date: Jun. 29, 2017

(54) IMAGING OF CREATINE KINASE ENZYME EXPRESSION IN CANCEROUS TISSUES

(71) Applicant: Sidra Medical and Research Center, Doha (QA)

(72) Inventors: Mohamma Haris, Doha (QA); Francesco Marincola, Palo Alto, CA (US)

(73) Assignee: Sidra Medical and Research Center, Doha (QA)

(21) Appl. No.: 15/325,059

(22) PCT Filed: Jul. 8, 2015
(86) PCT No.: PCT/US2015/039549

§ 371 Date: Jan. 9, 2017

Related U.S. Application Data
(60) Provisional application No. 62/022,488, filed on Jul. 9, 2014.

(51) Int. Cl. G01R 33/56; A61K 49/10; A61M 5/00; A61B 5/055; A61M 31/00

(52) U.S. Cl. CPC G01R 33/5601; A61B 5/055; A61M 31/005; A61M 5/007; A61K 49/10

(57) ABSTRACT

The present technology is directed to apparatuses, machines and methods for determining the level of expression of creatine kinase enzyme in cancerous tissues, as well as for determining malignancy and providing a cancer prognosis. The method is based on overexpression of creatine kinase in cancer tissue and enhanced transformation of injected phosphocreatine substrate into creatine as detectable by NMR.

### TECHNICAL FIELD

The present technology relates generally to apparatuses and methods for imaging expression of enzymes in cancerous tissues, in particular, creatine kinase enzyme.

### BACKGROUND

³¹Phosphorus MR spectroscopy (³¹PMRS) has been used to measure phosphorus metabolites including phosphocreatine (PCr) in vivo. It has also been also used to measure the metabolic flux from PCr to ATP. However, using ³¹PMRS, free creatine (Cr) cannot be measured since it measures only phosphorus metabolites. On the other hand proton MR spectroscopy (MRS) can only measure total creatine (PCr+Cr) and cannot differentiate between PCr and Cr. Proton MRS detects the metabolites based on the presence of aliphatic protons and both PCr and Cr have similar aliphatic protons and resonate on similar frequencies with proton MRS.

The chemical exchange saturation transfer (CEST) technique includes an enhancement mechanism that detects metabolite content based on exchange-related properties. The CEST technique involves selective saturation of compounds containing exchangeable protons or other molecules. After transfer of the saturation to bulk water, such compounds are detected through the change in the bulk water signal with enhanced sensitivity. In the CEST technique coupled with magnetic resonance imaging (MRI), the exchangeable protons on a solute pool can be irradiated by application of the radio-frequency (RF) pulse, and their saturated magnetization exchange with the bulk water leads to reduction in the bulk water signal in a concentration dependent manner.

That is, coupled with the RF pulse, one or tore magnets (a main magnet and several gradient magnets) can be applied to the sample to generate a magnetic field. When the RF pulse is turned off, the hydrogen protons slowly return to their natural alignment within the magnetic field and release the energy absorbed from the RE pulses. As they do this, they give off a signal that is transmitted to a computer and eventually to an output interface that can be read by a user.

Previously, the ability of the CEST technique to scan heart tissues has been investigated. However, to date, none of ³¹PMRS, ¹HMRS or CEST have been used to detect the creatine kinase enzyme expression in cancerous tissues. ³¹PMRS and ¹HMRS suffer from poor spatial resolution and low sensitivity; thus, these methods do not produce useful or meaningful results.

Thus, in various embodiments, the present technology is directed to apparatuses and methods involving, the use of the CEST technique for imaging the expression of creatine kinase enzyme in vivo and in vitro in cancerous tissues, including tumors.

### SUMMARY

In certain embodiments, the present technology is directed to a method of visually determining the level of expression of creatine kinase enzyme in a cancerous tissue, the method comprising the steps of: (a) exposing the cancerous tissue to phosphocreatine; and (b) irradiating the cancer tissue with a radio frequency (RF) pulse.

In other embodiments, the present technology is directed to a method of determining the malignancy of a cancerous tissue, the method comprising the steps of: (a) injecting phosphocreatine into the cancerous tissue; and (b) measuring the extent or rate of conversion of phosphocreatine into creatine in the cancerous tissue over a given time period; wherein the extent or rate of conversion of phosphocreatine into creatine in the cancerous tissue over the given time period is indicative of the malignancy of the cancerous tissue.

In other embodiments, the present technology is directed to method of providing a cancer prognosis, the method comprising the steps of: (a) injecting phosphocreatine intravenously into tissue known or thought to be cancerous; and (b) measuring the extent or rate of conversion of phosphocreatine into creatine in the cancerous tissue over a given time period. In certain embodiments, the extent or rate of conversion of phosphocreatine into creatine in the cancerous tissue is proportional to the level of expression of the creatine kinase enzyme, and wherein one or more of the extent or rate of conversion of phosphocreatine into creatine, or the level of expression of the creatine kinase enzyme, is an indicator of the cancer prognosis.

In other embodiments, the present technology is directed to an apparatus for monitoring the extent or rate of conversion of phosphocreatine to creatine in the body of a patient; the apparatus comprising: (a) a radio source capable of providing a radio frequency (RF) pulse to the body of the patient; and (b) a detector capable of measuring the extent or rate of conversion of phosphocreatine into creatine in the body of the patient over a given time period.

In other embodiments, the present technology is directed to a method of determining the expression of creatine kinase enzyme in a cancerous tissue, the method comprising the steps of:

(a) exposing the cancerous tissue to phosphocreatine;

(b) irradiating the cancer tissue with a radio frequency (RF) pulse;

(c) obtaining an image through magnetic resonance imaging (MRI) that indicates the level of expression of the creatine kinase enzyme over a given time period; and

(d) determining the extent or rate of conversion of phosphocreatine into creatine in the cancerous tissue based on the image, where the extent or rate of conversion of phosphocreatine into creatine in the cancerous tissue is proportional to the level of expression of the creatine kinase enzyme.

In other embodiments, the present technology is directed to a method of determining the level of expression of creatine kinase enzyme in cancerous tissue, the method comprising the steps of:

(a) identifying tissue thought to be cancerous;

(b) exposing the tissue to phosphocreatine;

(c) loading the tissue into an apparatus in proximity to a radio source and a magnet source;

(d) irradiating the tissue with a radio frequency (RF) pulse emitting from the radio source, and applying the magnetic source to the sample to produce a magnetic field;

(e) gathering data generated by step (d) to produce an image indicating the level of expression of the creatine kinase enzyme; and

(f) displaying the image on a visual output.

In other embodiments, the present technology is directed to machine comprising the following:

(a) a mechanism configured to hold a patient thought to have cancerous tissue or a sample of tissue thought to be cancerous, the tissue being in contact with phosphocreatine;

(b) a radio source configured to provide a radio frequency (RF) pulse to the tissue, and a magnet source configured to provide a magnetic field to the tissue;

(c) a timing mechanism configured to calculate a period of time elapsed from a starting point to an end point;

(d) a detector capable of measuring the extent or rate of conversion of phosphocreatine into creatine in the tissue over the period of time; and

(e) an output interface that displays the result of (d).

### DESCRIPTION OF THE DRAWINGS

FIG. 1 shows a flowchart in accordance with certain embodiments of the present technology.

FIG. 2 shows CEST imaging of cancer cells in an embodiment, in absence of phosphocreatine (PCr) and cultured with PCr.

FIG. 3 shows in vivo imaging of creatine kinase expression in an embodiment, by monitoring conversion of PCr to Cr through CEST.

### DETAILED DESCRIPTION

Magnetic resonance imaging (MRI) is a known method of imaging the cells of a patient. In its simplest description, a typical MRI technique produces an image of a selected body part or area by manipulating the magnetic spins of hydrogen atoms or protons in body parts such as fat and water molecules, and then measuring the signals of the manipulated magnetic spins. These measured signals can be analyzed and processed to provide images. An MRI system may be designed to generate different magnetic fields for imaging, including for example, a static magnetic field (B0) along a z-direction to polarize the magnetic spins, gradient fields along mutually orthogonal x, y, or z directions in a xyz coordinate system to spatially select a body part for imaging, and a radio frequency (RF) magnetic field (B1) to manipulate the spins.

The technology discussed herein is directed to the recent development of methods for scanning cancerous cells and tissues employing the CEST technique with MRI. Such technique is unexpectedly useful for detecting enzyme expression, in particular, creatine kinase enzyme expression in cancerous tissues such as tumors. CEST is a contrast enhancement technique that can permit indirect detection of metabolites with exchangeable protons. CEST agents can be useful as biomarkers of cancer and tumor growth.

Throughout the present disclosure, the terms “cancer,” “cancerous tissue,” “cancer cells,” “cancerous cells,” “tumor,” “tissue known to be cancerous” and “tissue thought to be cancerous” are used to refer to the same thing—one or more cells that exhibit signs of abnormal cell growth and spread in the body or are otherwise subject to the methods, machines or apparatuses herein for any reason. In certain embodiments, the apparatuses and methods discussed herein may be used for diagnosis or classification of cancer, and as such, the tissue may not turn out to be cancerous; thus, the terms “cancer,” “cancerous tissue,” “cancer cells,” “cancerous cells,” “tumor,” “tissue known to be cancerous” and “tissue thought to be cancerous” are also used herein to refer to any tissue that is thought to be cancerous, desired to be subjected to the methods, machines and apparatuses herein, and may or may not actually turn out to be cancerous. These can include, in various embodiments, tissue in the body of a patient (in vivo) as well as a sample of tissue that has been excised from the patient, for example, through a biopsy (in vitro). Throughout the present disclosure, all apparatuses, methods and machines contemplated herein can be used and performed in connection with both live patients as well as samples of tissue obtained from, and separate from, a patient.

Creatine kinase (CK) is an enzyme involved in cellular energy homeostasis through the creatine kinase reaction. Cells with higher cellular activity, such as brain and muscle cells, tend to have higher levels of CK activity. Higher levels of CK activity have also been observed in some primary cancerous tissues and metastatic lesions.

The brain isozyme of CK (CK-BB) is associated with cancer. It has been shown that the up-regulation of CK-BB is associated with the malignant transformation. It has also been shown that breast cancer patients with high tumor levels of CK-BB tend to have a higher risk for death compared to those with low CK-BB.

So far, only immunohistochemical analysis has been performed to detect the CK expression in cancerous tissues. This is primarily because quantification of CK enzymes by biochemical methods is invasive and requires tissue excision and prolonged analysis. Before now there has been no known method that can provide high resolution imaging of CK expression in vivo, in cancerous tissues.

Detection of CK expression in vivo may provide a diagnostic marker for tumor malignancy as well as an indication of a patient's response to treatment. Efforts have been made to monitor the CK reactions by measuring the CK metabolites using ³¹Phosphorus magnetic resonance spectroscopy (³¹P MRS).

The chemical exchange saturation transfer (CEST) technique has been used to measure the free creatine level in muscular tissue and the changes in creatine level following calf muscles exercise and myocardium infarction. However, the CEST technique has been shown to be additionally useful for detecting creatine kinase expression, which in certain embodiments provides a diagnostic marker for tumor malignancy as well as an indication of the patient's response to treatment.

In the CEST technique, exchangeable solute protons that resonate at a frequency different from that of bulk water protons are selectively saturated using radio frequency (RF) irradiation. These saturated protons are then transferred to bulk water when the solute protons exchange with water protons and the water signal becomes attenuated. Since the solute protons are present in bulk water in a relatively low concentration, a single transfer or saturation is generally insufficient to show any discernible effect on water protons. However, because the water pool is much larger than the saturated solute proton pool, each exchanging saturated solute proton is replaced by a nonsaturated water proton, which is then saturated again. Thus, if the exchange rate is fast enough, the continued RF irradiation will lead to a cumulative enhancement effect, which will eventually be detectable such that the solute's presence can be imaged with MRI. Thus, the CEST technique is an advantageous way to gather imaging information about solutes that are present in low concentrations.

The resultant MRI images and data provide information including identification and verification of the cancerous tissue, the extent and spread of the cancer, the size of the tumor, other characteristics of the tumor such as the density and depth of penetration into the patient's organs or tissues. This information may be used to predict factors such as future predicted growth of the cancerous tissue, the malignancy of the cancerous tissue, and the prognosis of the patient's cancer.

As mentioned above, the CK enzyme catalyzes the reversible conversion from phosphocreatine to creatine. In certain embodiments, the present technology is directed to methods based on the CEST effect from amine proton (—NH₂) of creatine to image CK expression in vivo in cancerous tissues. Amine protons are essentially labile protons that are in continuous exchange with the bulk water protons; by exploiting these amine protons, the metabolites can be imaged using the CEST technique. The exchange rate of amine protons differs from metabolite to metabolite. Phosphocreatine in its native form does not exhibit CEST effect—that is, the CEST technique cannot be used to image it. This is because the amine protons present in phosphocreatine have been found to depict a very slow exchange rate compared to those present in creatine, and thus do not provide appreciable CEST contrast. However, the amine protons of creatine have been discovered to show an appreciable CEST contrast. In certain embodiments of the present technology, CEST is used to map the free creatine separately from phosphocreatine. In certain embodiments, this mapping is done at high resolution.

In certain embodiments herein, phosphocreatine conversion into creatine in cancerous tissues due to high level of CK enzyme expression can enhance the CEST contrast from a tumor region. Phosphocreatine in amounts of up to several millimolar without any deleterious effects. The rate of cleavage of phosphocreatine into creatine in tumor tissues will be proportional to the extent of CK enzyme expression, and thus, is indicative of the expression of the CK enzyme and malignancy of the cancer. In certain embodiments one or more of the extent or rate of conversion of phosphocreatine into creatine, or the level of expression of the creatine kinase enzyme, is an indicator of the malignancy of the cancerous tissue; for example, in direct proportion, or in a proportion that can be mathematically shown or predicted.

Higher expression of CK in malignant tumors will result in more conversion of phosphocreatine in to creatine. In certain embodiments, using the CEST technique the extent or rate of phosphocreatine to creatine can be monitored non-invasively. Thus, this can be used as a biomarker, to observe the CK enzyme activity and expression in vivo, to monitor the tumor progression, and to predict or calculate tumor malignancy, cancer prognosis and treatment efficiency and efficacy (degree of success or failure). Such methods and processes can be used in diagnosis and treatment monitoring of any cancer known to afflict mammals, including but not limited to breast, prostate, ovarian, brain, lung, stomach, colorectal, liver, pancreatic and the like.

In certain embodiments, the radio frequency (RF) pulse can be provided at different frequency offsets. In certain embodiments, to detect the conversion of phosphocreatine to creatine, an RF pulse was applied at a frequency offset of about 1.8 ppm for a period of about 1 to about 3 seconds.

In certain embodiments, the methods herein include monitoring of the extent or rate of conversion of phosphocreatine to creatine over a given period of time. As used herein, “monitoring” includes discrete monitoring and continuous monitoring.

For example, in the case of discrete monitoring, the present technology contemplates exposing the cancerous tissue to the phosphocreatine, taking a first measurement, then waiting a proscribed period of time, and then taking a second measurement in order to ascertain the extent of conversion from phosphocreatine to creatine in that proscribed period of time. These steps may be followed optionally by any number of subsequent measurements in accordance with the same procedure.

As used herein, the terms “exposing,” “contacting” or “delivering” are used interchangeably in the context of bringing the phosphocreatine into contact with the cancerous tissue, and all refer to any form of contact between the two.

In the case of continuous monitoring, the monitoring includes ongoing monitoring of the extent or rate (or both extent and rate) of conversion of phosphocreatine to creatine over a given period of time. For example, in MRI, generally only the extent of the conversion of phosphocreatine to creatine over a given period of time can be determined. However, in the related process of functional MRI (FMRI), both the extent and rate of conversion can be determined. The present technology contemplates applications with both traditional MRI and FMRI.

In in various embodiments, useful information can be gathered over a period of minutes, hours, days or weeks, for example, about 1 (60 minutes) to about 3 hours (180 minutes), about 45 to about 240 minutes or about 1 day.

In certain embodiments, the present technology contemplates an apparatus for monitoring the conversion of phosphocreatine to creatine in the body of a patient. The apparatus may comprise a radio source capable of providing a radio frequency (RF) pulse to the body of the patient; and a detector capable of measuring the rate of conversion of phosphocreatine into creatine in the body of the patient. In certain embodiments, the radio source may be incorporated into the MRI machine that is capable of scanning a cancerous tissue such as a tumor in vivo or in vitro; in other embodiments, the radio source and the MRI machine may be separate. In certain embodiments, the methods herein comprise the following steps:

(1) The cancerous tissue (or tissue that is thought to be cancerous) is exposed to the phosphocreatine (in certain embodiments by intravenous injection of the phosphocreatine, for example, intravenously injected in a manner such that the phosphocreatine upon injection will reach the cancerous tissue through the bloodstream, and the cancerous cells in the tissue will engulf the phosphocreatine;

(2) The tissue is then irradiated with the RF pulse for a period of about 1 to about 3 seconds, resulting in a visual indication of the level of expression of the creatine kinase enzyme. As used herein, “visual indication” refers to the image that is generated as a result of the RF radiation, in certain embodiments through magnetic resonance imaging. In various embodiments, this visual indication itself provides valuable information to the investigator about the cancerous (or thought to be cancerous) tissue's identity, rate of growth, malignancy, aggressiveness, prognosis or other physical characteristics; or may be used to generate an image via MRI;

(3) An MRI scan is taken of the cancerous tissue, resulting in an image that shows tissue's identity, rate of growth, malignancy, aggressiveness, prognosis or other physical characteristics.

(4) Information regarding the tumor's identity, rate of growth, malignancy, aggressiveness, prognosis and or other physical characteristics can then be processed and ascertained, either as part of the same apparatus, or offline by a dedicated individual or computer that can determine the change in the CEST contrast.

In certain embodiments, the cancerous tissue is exposed to the phosphocreatine by any of the following methods:

intravenous injection of the phosphocreatine into the body of a patient, which permits the phosphocreatine to travel through the body to the site of the tumor or cancerous cells; or

injection of the phosphocreatine directly to or proximate to the site of the tumor or cancerous cells in the body of the patient; or

injection of the phosphocreatine directly into a sample of cells separate from the body of a patient (that is, tissue that has been removed from the patient, as in a biopsy); or

any other way of contacting the phosphocreatine with the tumor or cancerous cells, as in a petri dish or in the patient's body, or otherwise in vitro or in vivo.

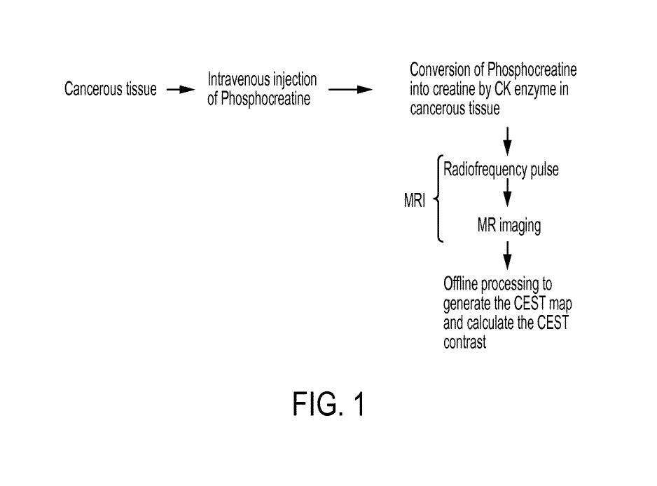

As can be seen in FIG. 1, one exemplary and non-limiting method is illustrated by the following steps: phosphocreatine is contacted 3 with the cancerous tissue 1. The RF pulse is applied from the RF source 2, resulting in a visual indication 4 of the level of expression of the kinase enzyme. This visual indication may be any of the following: a chart, a graph, numerical data, visual information in the form of images in video or photographic form (for example, a time lapse video, a microscope slide, or any other format that is scientifically acceptable and customarily used). The visual indication may then be subjected to magnetic resonance imaging through an MRI machine 5, producing an MRI image 6 that provides information, including but not limited to the tissue's identity, rate of growth, malignancy, aggressiveness, prognosis or other physical characteristics.

In certain embodiments, the changes to the CK signal may be determined over time, with the changes over time being indicative of characteristics of the cancerous tissue. For example, if the signal strength increases over time, this can indicate, among other conditions, a higher degree of malignancy or an aggressively growing cancer. If the signal strength decreases over time, this can indicate, among other conditions, a lower degree of malignancy, a less aggressive cancer (or absence of cancer) or a more optimistic prognosis for the patient.

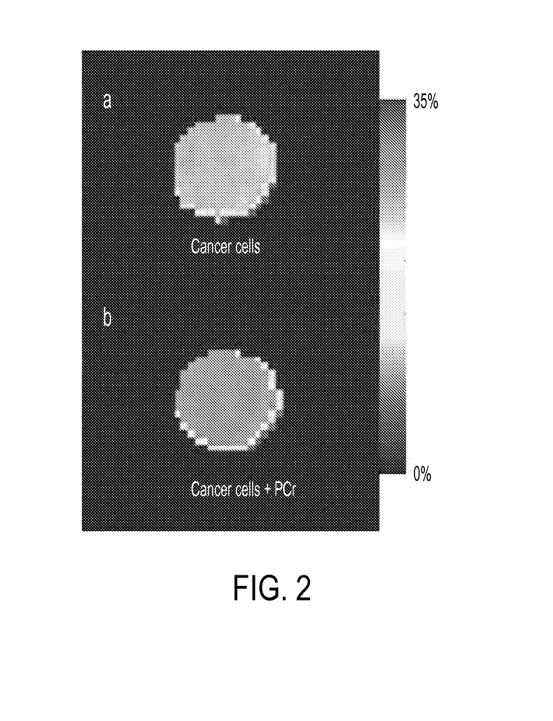

FIG. 2 shows CEST imaging of cancer cells, as follows: “a” is CEST map of Cancer cells in absence of Phosphocreatine (PCr); whereas “b” shows the same cell line cultured with PCr for 1 hour, indicating an elevation of about 16% of the CEST contrast over cancer cells without PCr. The creatine kinase (CK) expression in cancer cells cleaves the PCr into creatine (Cr), and can be responsible for the increase in CEST contrast from creatine amine protons.

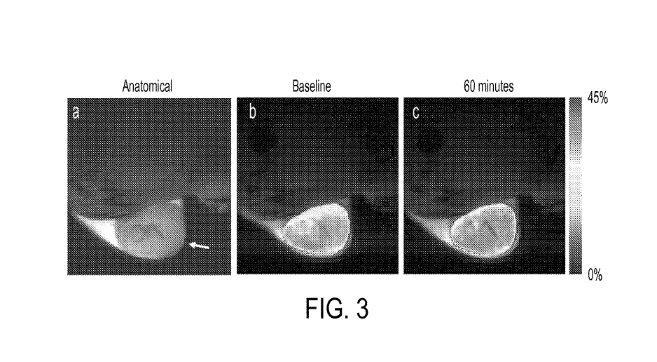

FIG. 3 shows in vivo imaging of creatine kinase expression by monitoring conversion of PCr to Cr through CEST. “a” is an anatomical CEST weighted image, which shows the tumor as a hyperintense region (arrow). “b” is the base line CEST map from the same slice. “c” is a CEST map of the same slice at 60 minutes following tail vein injection of PCr. It demonstrates that an about 11% increase in CEST contrast was observed at 60 minutes post infusion, which is due to conversion of PCr into Cr by the CK enzyme expressed in the tumor.

Thus, in certain embodiments, an increase of about 10 to about 20%, about 11%, about 15%, about 16% or about 20% increase in CEST contrast was observed at 60 minutes post infusion, due to conversion of PCr into Cr by the CK enzyme expressed in tumor.

The current technology may help in staging the tumor aggressiveness—that is, assessing the extent to which a tumor has spread. The activity of creatine kinase enzyme can be monitored non-invasively using the currently discussed methods. In certain embodiments, this could be easily implemented on a clinical MRI scanner for routine clinical examination of tumor biology as well as to monitor the therapeutic response.

The current technology can also be very useful for applications such as targeted drug delivery, monitoring the therapeutic responses in human and animal models of tumors and the development of drugs and therapeutics.

In certain embodiments, phosphocreatine can be industrialized as a MRI contrast agent to map the creatine kinase enzyme activity in cancerous tissues at high resolution.

In certain embodiments, the aggressiveness of cancerous tissues can be estimated in vivo or in vitro based on CK expression. The technology herein can be used to differentiate benign vs. malignant cancers, as well as to predict cancer prognosis, drug development and therapy, and monitoring in animal model studies of different cancers. The technology can also be used to image tumor tissue, in clinical diagnosis of tumors, and also to monitor therapeutic efficacy. For example, the technology can provide a non-invasive way to ascertain whether a treatment is working.

Although the present technology has been described in relation to particular embodiments thereof, these embodiments and examples are merely exemplary and not intended to be limiting. Many other variations and modifications and other uses will become apparent to those skilled in the art. The present technology should, therefore, not be limited by the specific disclosure herein, and may be embodied in other forms not explicitly described here, without departing from the spirit thereof.

1. A method of determining the level of expression of creatine kinase enzyme in a cancerous tissue, the method comprising the steps of:
(a) exposing the cancerous tissue to phosphocreatine; and
(b) irradiating the cancerous tissue with a radio frequency (RF) pulse.

2. The method of claim 1, wherein the cancerous tissue is exposed to the phosphocreatine by any of the following methods: intravenous injection of the phosphocreatine into the body of a patient, or injection of the phosphocreatine directly into the site of the cancerous tissue.

3. The method of claim 1, wherein the cancerous tissue is a tumor.

4. The method of claim 1, wherein the visual indication is created with magnetic resonance imaging.

5. A method of determining the malignancy of a cancerous tissue, the method comprising the steps of:
(a) injecting phosphocreatine into the cancerous tissue; and
(b) measuring the extent or rate of conversion of phosphocreatine into creatine in the cancerous tissue over a given time period;
wherein the extent or rate of conversion of phosphocreatine into creatine in the cancerous tissue over the given time period is indicative of the malignancy of the cancerous tissue.

6. A method of providing a cancer prognosis, the method comprising the steps of:
(a) injecting phosphocreatine intravenously into tissue known or thought to be cancerous; and
(b) measuring the extent or rate of conversion of phosphocreatine into creatine in the cancerous tissue over a given time period.

7. An apparatus for monitoring the extent or rate of conversion of phosphocreatine to creatine in the body of a patient; the apparatus comprising:
(a) a radio source capable of providing a radio frequency (RF) pulse to the body of the patient; and
(b) a detector capable of measuring the extent or rate of conversion of phosphocreatine into creatine in the body of the patient over a given time period.

8. The apparatus of claim 7, further comprising:
(c) an imaging component that generates an image of a portion of the body of the patient.

9. The apparatus of claim 7, wherein the image is generated with magnetic resonance.

10. The apparatus of claim 7, wherein the given time period is about 1 hour to about 24 hours.

11. The method of claim 1, further comprising the steps of:
(c) obtaining an image through magnetic resonance imaging (MRI) that indicates the level of expression of the creatine kinase enzyme over a given time period; and
(d) determining the extent or rate of conversion of phosphocreatine into creatine in the cancerous tissue based on the image, where the extent or rate of conversion of phosphocreatine into creatine in the cancerous tissue is proportional to the level of expression of the creatine kinase enzyme.

12. The method of claim 11, wherein one or more of the extent or rate of conversion of phosphocreatine into creatine, or the level of expression of the creatine kinase enzyme, is an indicator of the cancer prognosis.

13. The method of claim 11, wherein the extent or rate of conversion of phosphocreatine into creatine over the given time period of 60 minutes is about 10 to about 20% greater than that of the cancer cells in the absence of phosphocreatine.

14. A method of determining the level of expression of creatine kinase enzyme in cancerous tissue, the method comprising the steps of:
(a) identifying tissue thought to be cancerous;
(b) exposing the tissue to phosphocreatine;
(c) loading the tissue into an apparatus in proximity to a radio source and a magnet source;
(d) irradiating the tissue with a radio frequency (RF) pulse emitting from the radio source, and applying the magnetic source to the sample to produce a magnetic field;
(e) gathering data generated by step (d) to produce an image indicating the level of expression of the creatine kinase enzyme; and
(f) displaying the image on a visual output.

15. (canceled)

16. The apparatus of claim 7, further comprising:
(c) a mechanism configured to hold a patient thought to have cancerous tissue or a sample of tissue thought to be cancerous, the tissue being in contact with phosphocreatine;
(d) a magnet source configured to provide a magnetic field to the tissue;
(e) a timing mechanism configured to calculate a period of time elapsed from a starting point to an end point;
(f) a detector capable of measuring the extent or rate of conversion of phosphocreatine into creatine in the tissue over the period of time; and
(g) an output interface that displays the result of (d).
